(19) United States
(12) Patent Application Publication
(10) Pub. No.: US 2026/0225087 A1
(43) Pub. Date: Aug. 6, 2026

(54) CATALYSTS FOR SOLVOLYSIS OF POLYESTER

(71) Applicant: Agency for Science, Technology and Research, Singapore (SG)

(72) Inventors: Cun Wang, Singapore (SG); Jui Hsien Michelle Ong, Singapore (SG); Martin Van Meurs, Singapore (SG); Ludger Paul Stubbs, Singapore (SG)

(73) Assignee: Agency for Science, Technology and Research, Singapore (SG)

(21) Appl. No.: 19/149,747

(22) PCT Filed: Jan. 26, 2024
(86) PCT No.: PCT/SG2024/050051

§ 371 Date: Jul. 21, 2025

(30) Foreign Application Priority Data
Jan. 27, 2023 (SG) 10202300221W

(51) Int. Cl. B01J 31/02; C07C 67/03; C08J 11/28

(52) U.S. Cl. CPC B01J 31/0268; C07C 67/03; C08J 11/28; B01J 2231/005; C08J 2367/02

(57) ABSTRACT

Disclosed herein is a process and a catalysis for the solvolysis of a polyester. The process comprises (a) mixing the polyester with a solvolysis reagent and the catalyst, wherein the catalyst comprises: a cation of formula Ia: [PR₁R₂R₃R₄]+, wherein each of R₁, R₂, R₃ and R₄ is independently selected from a linear or branched C₁ to C₂₀ alkyl group; or wherein R₁ and R₂ and/or R₃ and R₄ together form an alkylene chain —(CH₂)ₚ— where p is from 3 to 6, such that R₁ and R₂ and/or R₃ and R₄ form, together with the phosphorous atom that they are attached to, a cyclic ring; and an anion; and (b) heating the mixture to a predetermined temperature.

### FILED OF INVENTION

The present invention provides a process for the solvolysis of a polyester using a phosphonium catalyst and a phosphonium catalyst.

### BACKGROUND

The listing or discussion of a prior-published document in this specification should not necessarily be taken as an acknowledgement that the document is part of the state of the art or is common general knowledge.

The recycling of polyesters, including polyethylene terephthalate (PET), from plastic waste can be done mechanically or chemically. Only high quality, transparent PET waste is suitable for mechanical recycling. For contaminated PET waste, chemical recycling is the method of choice. The most straightforward way is solvolysis, which involves breaking down the ester bonds in the PET with a reactive solvent like water (hydrolysis), or alcohols (methanolysis or glycolysis). The advantage of glycolysis is that the resulting diester of terephthalic acid, BHET, can be directly used for the repolymerization to PET in commercial plants. The challenge is to find catalysts which are both active and stable at high reaction temperatures (190° C.) and in the presence of contaminants from the plastic waste. To avoid contamination by trace metals, the use of organo-catalysts for the glycolysis of PET has found widespread interest. A range of organo-catalysts has been described in literature, with 1,5,7-triazabicyclo[4.4.0]dec-5-ene (TBD), a guanidine type catalyst, being a prominent benchmark. However, the lack of thermal stability and the comparatively high cost of TBD are disadvantages that warrant further catalyst development work.

Therefore, there exists a need for alternative and/or improved catalysts and processes for the solvolysis of a polyester.

### SUMMARY

Aspects and embodiments of the current invention will now be described by reference to the following numbered clauses.

1. A process for the solvolysis of a polyester, the process comprising:
(a) mixing the polyester with a solvolysis reagent and a catalyst, wherein the catalyst comprises:
a cation of formula Ia:

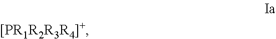

wherein each of R₁, R₂, R₃ and R₄ is independently selected from a linear or branched C₁ to C₂₀ alkyl group; or
wherein R₁ and R₂ and/or R₃ and R₄ together form an alkylene chain-(CH₂)ₚ— where p is from 3 to 6, such that R₁ and R₂ and/or R₃ and R₄ form, together with the phosphorous atom that they are attached to, a cyclic ring; and
an anion; and
(b) heating the mixture to a predetermined temperature.

2. The process according to Clause 1, wherein each of R₁, R₂, R₃ and R₄ is independently a linear or branched C₂ to C₁₀ alkyl group, such as a linear or branched C₃ to C₈ alkyl group.

3. The process according to Clause 1, wherein the cation is a tetrabutylphosphonium ion.

4. The process according to Clause 1, wherein the cation is

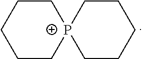 [SMILES: C1CC[P+]2(CC1)CCCCC2]

5. The process according to any one of the preceding clauses, wherein the anion is selected from the group consisting of a hydroxide, a halide, an alkoxide, a phenoxide, a carboxylate, a carbonate, a bicarbonate and a sulfonate, optionally wherein the anion is selected from the group consisting of an alkoxide, a phenoxide, a carboxylate, a carbonate, and a bicarbonate, such as an alkoxide, a phenoxide, or a carboxylate.

6. The process according to Clause 5, wherein:
(ai) when the anion is a halide, the halide is selected from chloride and bromide;
(aii) when the anion is an alkoxide, the alkoxide has a formula IIa:

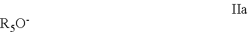

wherein R₅ is a linear or branched C₁ to Ce alkyl group;
(aiii) when the anion is a phenoxide, the phenoxide has a formula IIb:

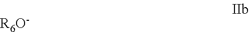

wherein Rₑ is an aryl group which is unsubstituted or substituted by one or more linear or branched C₁ to C₄ alkyl groups,
each the linear or branched C₁ to C₄ alkyl group is unsubstituted or substituted by a second aryl group which is unsubstituted or substituted by a hydroxyl group, optionally wherein the phenoxide forms a hydrogen bond with a second protonated phenoxide to form a dimer;
(aiv) when the anion is a carboxylate, the carboxylate has a formula IIc:

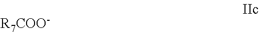

wherein R₇ is selected from a linear or branched C₁ to C₆ alkyl group and an aryl group which is unsubstituted or substituted by one or more substituents selected from a linear or branched C₁ to C₄ alkyl group and a carboxylate group, optionally wherein the anion is p-terephthalate;
(av) when the anion is a sulfonate, the sulfonate has a formula IId:

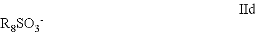

wherein R₈ is selected from a linear or branched C₁ to C₆ alkyl group and an aryl group which is unsubstituted or substituted by one or more linear or branched C₁ to C₄ alkyl groups.

7. The process according to any one of the preceding clauses, wherein the anion is selected from the group consisting of:

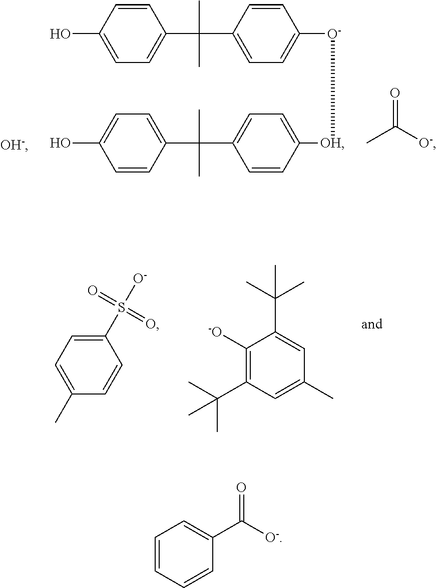

8. The process according to any one of the preceding clauses, wherein the anion is selected from the group consisting of:

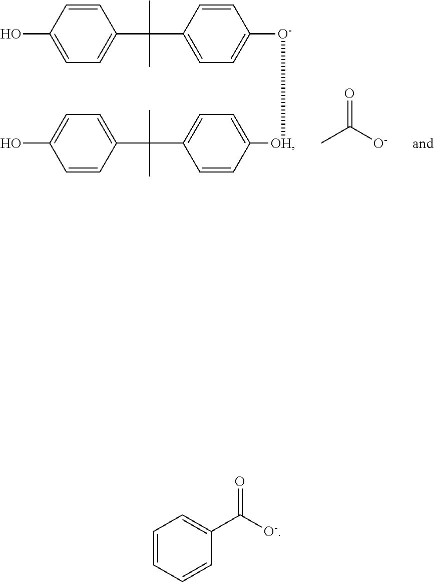

9. The process according to any one of the preceding clauses, wherein the predetermined temperature is from 180 to 240° C.

10. The process according to any one of the preceding clauses, wherein the solvolysis reagent is selected from the group consisting of water, an alcohol, an amine and ammonia, optionally wherein the alcohol is selected from a group consisting of methanol, ethanol, sorbitol 1,3-propanediol, 1,4-butanediol, 1,6-hexanediol and ethylene glycol.

11. The process according to any one of the preceding clauses, wherein the solvolysis reagent is ethylene glycol.

12. The process according to one of the preceding clauses, wherein the polyester is an aliphatic polyester or aromatic polyester, optionally wherein the aromatic polyester is selected from the group consisting of polyethylene terephthalate (PET), polybutylene terephthalate (PBT), polyhexamethylene terephthalate (PHT), polypropylene terephthalate (PPT).

13. The process according to one of the preceding clauses, wherein the polyester is polyethylene terephthalate (PET).

14. A catalyst for the solvolysis of a polyester, the catalyst comprises:
a cation of formula Ia:

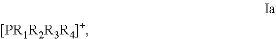

wherein R₁, R₂, R₃ and R₄ are independently a linear or branched C₁ to C₂₀ alkyl group; or
wherein R₁ and R₂ and/or R₃ and R₄ together form an alkylene chain-(CH₂)ₚ— where p is from 3 to 6, such that R₁ and R₂ and/or R₃ and R₄ form, together with the phosphorous atom that they are attached to, a cyclic ring; and
an anion,
wherein the anion is selected from the group consisting of an alkoxide, a phenoxide, a carbonate, a bicarbonate and a sulfonate, optionally wherein the anion is selected from the group consisting of an alkoxide, a phenoxide, a carboxylate, a carbonate, and a bicarbonate, such as an alkoxide, a phenoxide, or a carboxylate.

15. The catalyst according to Clause 14, wherein:
(a) when the anion is an alkoxide, the alkoxide has a formula IIa:

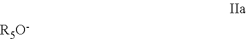

wherein R₅ is a linear or branched C₁ to Ce alkyl group;
(b) when the anion is a phenoxide, the phenoxide has a formula IIb:

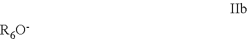

wherein Rₑ is an aryl group which is unsubstituted or substituted by one or more linear or branched C₁ to C₄ alkyl groups,
each the linear or branched C₁ to C₄ alkyl group is unsubstituted or substituted by a second aryl group which is unsubstituted or substituted by a hydroxyl group, optionally wherein the phenoxide forms a hydrogen bond with a second protonated phenoxide to form a dimer;
(c) when the anion is a sulfonate, the sulfonate has a formula IId:

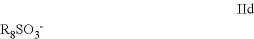

wherein R₈ is selected from a linear or branched C₁ to C₆ alkyl group and an aryl group which is unsubstituted or substituted by one or more linear or branched C₁ to C₄ alkyl groups.

16. The catalyst according to Clause 14 or Clause 15, wherein the anion is selected from the group consisting of:

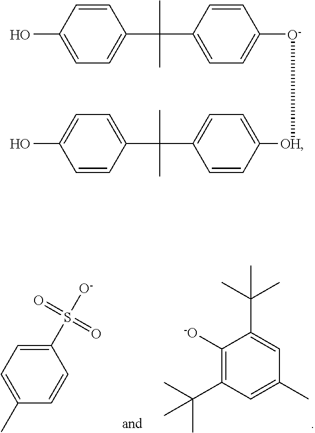

17. The catalyst according to any one of Clauses 14 to 16, wherein the anion is

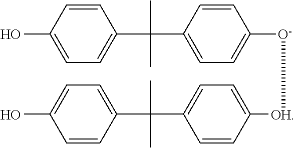

18. The catalyst according to any one of Clauses 14 to 17, wherein each of R₁, R₂, R₃ and R₄ is independently a linear or branched C₂ to C₁₀, such as a linear or branched C₃ to C₅ alkyl group.

19. The catalyst according to any one of Clauses 14 to 18, wherein the cation is a tetrabutylphosphonium ion.

19. The catalyst according to any one of Clauses 14 to 17, wherein the cation is

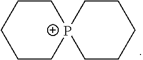

### DRAWINGS

FIG. 1 includes ³¹P NMR spectra of CAT28 TBP-BPA₂ from the thermal stability study in accordance to Example 4.

FIG. 2 includes ¹H NMR spectra of CAT28 TBP-BPA₂ from the thermal stability study in accordance to Example 4.

FIG. 3 includes ³¹P NMR spectra of CAT30 TBP-acetate from the thermal stability study in accordance to Example 4.

FIG. 4 includes ¹H NMR spectra of CAT30 TBP-acetate from the thermal stability study in accordance to Example 4.

FIG. 5 includes ³¹P NMR spectra of CAT37 TBP-benzoate from the thermal stability study in accordance to Example 4.

FIG. 6 includes ¹H NMR spectra of CAT37 TBP-benzoate from the thermal stability study in accordance to Example 4.

FIG. 7 includes ¹H NMR spectra of TBD from the thermal stability study in accordance to Example 4.

### DESCRIPTION

It has been surprisingly found that a new type of organocatalysts can be used for the solvolysis of polyesters, including glycolysis of polyethylene terephthalate (PET) (Scheme 1). The catalysts are superior in performance and possess high stability at high reaction temperatures (at least 190° C.) and in the presence of contaminants from plastic waste compared to conventional catalysts, including 1,5,7-triazabicyclo[4.4.0]dec-5-ene (TBD), which is a prominent benchmark catalyst used for solvolysis.

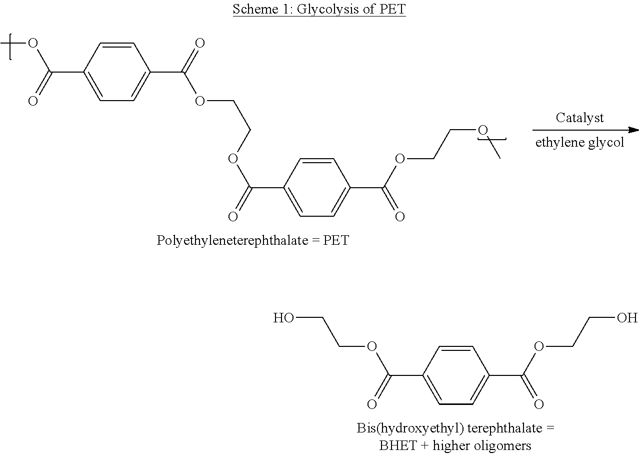

Thus, in a first aspect of the invention, there is provided a process for the solvolysis of a polyester, the process comprising:
(a) mixing the polyester with a solvolysis reagent and a catalyst, wherein the catalyst comprises:
a cation of formula Ia:

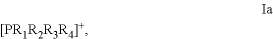

wherein each of R₁, R₂, R₃ and R₄ is independently a linear or branched C₁ to C₂₀ alkyl group; or
wherein R₁ and R₂ and/or R₃ and R₄ together form an alkylene chain-(CH₂)ₚ— where p is from 3 to 6, such that R₁ and R₂ and/or R₃ and R₄ form, together with the phosphorous atom that they are attached to, a cyclic ring; and
an anion; and
(b) heating the mixture to a predetermined temperature.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, “alkyl” refers to an unbranched or branched, cyclic, saturated hydrocarbyl radical, which may be substituted or unsubstituted.

In some embodiments of the invention that may be mentioned herein, each of R₁, R₂, R₃ and R₄ may be independently a linear or branched C₂ to C₁₀ alkyl group, such as a linear or branched C₃ to C₈ alkyl group. In more particular embodiments, wherein the cation may be a tetrabutylphosphonium ion.

In some embodiments of the invention that may be mentioned herein, the cation may be a cyclic phosphonium cation. In more particular embodiments, the cation may be a bicyclic phosphonium cation. In more particular embodiments, the cation may be

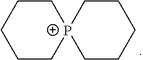

In some embodiments of the invention that may be mentioned herein, the anion may be selected from the group consisting of a hydroxide, a halide, an alkoxide, a phenoxide, a carboxylate, a carbonate, a bicarbonate and a sulfonate. In more particular embodiments, the anion may be selected from the group consisting of an alkoxide, a phenoxide, a carboxylate, a carbonate, a bicarbonate, for example, an alkoxide, a phenoxide, or a carboxylate.

When the anion is a halide, the halide may be selected from chloride and bromide.

When the anion is an alkoxide, the alkoxide has a formula IIa:

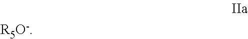

In some embodiments of the invention, R₅ may be a linear or branched C₁ to C₆ alkyl group.

When the anion is a phenoxide, the phenoxide has a formula IIb:

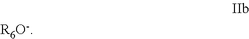

In some embodiments of the invention, R₆ may be an aryl group which may be unsubstituted or substituted by one or more linear or branched C₁ to C₄ alkyl groups, each the linear or branched C₁ to C₄ alkyl group may be unsubstituted or substituted by a second aryl group which may be unsubstituted or substituted by a hydroxyl group. In more particular embodiments, the phenoxide may form a hydrogen bond with a second protonated phenoxide to form a dimer. In more particular embodiments, the phenoxide may be

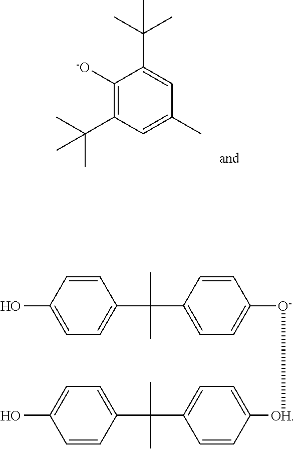

Unless otherwise stated, the term “aryl” when used herein includes C₆-C₁₄ (such as C₆-C₁₀) aryl groups. Such groups may be monocyclic, bicyclic or tricyclic and have between 6 and 14 ring carbon atoms, in which at least one ring is aromatic. The point of attachment of aryl groups may be via any atom of the ring system. However, when aryl groups are bicyclic or tricyclic, they are linked to the rest of the molecule via an aromatic ring. C₆-14 aryl groups include phenyl, naphthyl and the like, such as 1,2,3,4-tetrahydronaphthyl, indanyl, indenyl and fluorenyl. Embodiments of the invention that may be mentioned include those in which aryl is phenyl.

When the anion is a carboxylate, the carboxylate has a formula IIc:

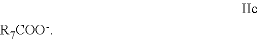

In some embodiments of the invention, R₇ may be selected from a linear or branched C₁ to C₆ alkyl group and an aryl group which may be unsubstituted or substituted by one or more substituents selected from a linear or branched C₁ to C₄ alkyl group and a carboxylate group.

In more particular embodiments, the carboxylate may be

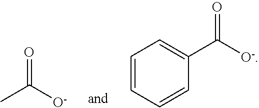

In more particular embodiments, the carboxylate may be p-terephthalate.

When the anion is a sulfonate, the sulfonate has a formula IId:

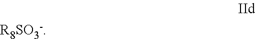

In some embodiments of the invention, R₈ may be selected from a linear or branched C₁ to C₆ alkyl group and an aryl group which may be unsubstituted or substituted by one or more substituents selected from a linear or branched C₁ to C₄ alkyl group. In more particular embodiments, the sulfonate may be

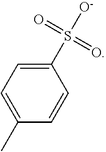

In some embodiments of the invention that may be mentioned herein, the predetermined temperature that the mixture of the catalyst and polyester is being heated to during the process of solvolysis may be from 180 to 240° C. Advantageously, the catalysts of the present invention have high thermal stability and are able to withstand temperatures of at least 190° C. compared to conventional catalysts used for the solvolysis of polyester.

For the avoidance of doubt, it is explicitly contemplated that where a number of numerical ranges related to the same feature are cited herein, that the end points for each range are intended to be combined in any order to provide further contemplated (and implicitly disclosed) ranges. Thus, in relation to the above related numerical ranges, there is disclosed a predetermined temperature of:
from 180 to 190° C., from 180 to 200° C., from 180 to 210° C., from 180 to 220° C., from 180 to 230° C., from 180 to 240° C.;
from 190 to 200° C., from 190 to 210° C., from 190 to 220° C., from 190 to 230° C., from 190 to 240° C.;
from 200 to 210° C., from 200 to 220° C., from 200 to 230° C., from 200 to 240° C.;
from 210 to 220° C., from 210 to 230° C., from 210 to 240° C.;
from 220 to 230° C., from 220 to 240° C.; and
from 230 to 240° C.

When used herein, the term “about”, in the context of temperature, may mean a variation of +5% of the stated value, more typically +/−4% of the stated value, more typically +3% of the stated value, more typically, +/−2% of the stated value, even more typically +1% of the stated value, and even more typically +/−0.5% of the stated value.

In some embodiments of the invention that may be mentioned herein, the solvolysis reagent may be any suitable reactive solvent for use in the breakdown of a polyester. In some embodiments of the invention, the solvolysis reagent may be selected from a group consisting of water, an alcohol, an amine and ammonia. In more particular embodiments, the alcohol may be selected from a group consisting of methanol, ethanol, sorbitol 1,3-propanediol, 1,4-butanediol, 1,6-hexanediol and ethylene glycol. In more particular embodiments, the solvolysis reagent may be ethylene glycol.

In some embodiments of the invention that may be mentioned herein, the polyester may be any suitable polymer that contains an ester functional group in every repeating unit of the main chain. In some embodiments of the invention, the polyester may be an aliphatic polyester or aromatic polyester. In more particular embodiments, the aromatic polyester is selected from the group consisting of polyethylene terephthalate (PET), polybutylene terephthalate (PBT), polyhexamethylene terephthalate (PHT), polypropylene terephthalate (PPT). In more particular embodiments, the aromatic polyester may be polyethylene terephthalate.

In a second aspect of the invention, there is provided a catalyst for the solvolysis of a polyester. The catalyst is identical to the catalyst described in relation to the process for the solvolysis of a polyester in the first aspect of invention hereinbefore. As such, discussion of the catalyst to this aspect of invention is omitted for the sake of brevity.

Further aspects and embodiments of the invention will now be discussed by reference to the following non-limiting examples below.

### EXAMPLES

### Example 1: Synthesis of Tetrabutylphosphonium (TBP) Catalysts

A range of phosphonium catalysts were synthesized from tetrabutylphosphonium hydroxide and tested for the glycolysis of PET (see Table 1). Tetrabutylphosphonium hydroxide was purchased from a commercial source and used without further purification. A benchmark catalyst, 1,5,7-triazabicyclo[4.4.0]dec-5-ene (TBD) (see Table 2) was used for comparison with the TBP catalysts.

TABLE 1
Exemplified TBP catalysts

| | | | | | Catalyst |
|---|---|---|---|---|---|
| | Cation | | Counterion | | Code |
| 1 2 | Tetrabutyl phosphonium | 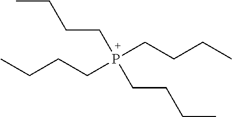 | Hydroxide BPA₂ | OH⁻ 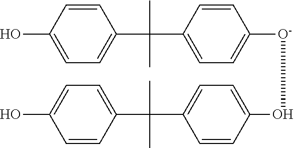 | CAT26 CAT28 |
| 3 | | | Acetate | 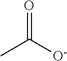 | CAT30 |
| 4 | | | Butylated hydroxytoluene | 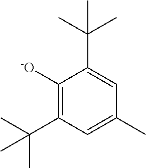 | CAT32 |
| 5 | | | Tosylate | 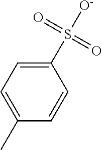 | CAT35 |
| 6 | | | Benzoate | 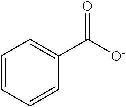 | CAT37 |

TABLE 2
Benchmark catalyst

| | Benchmark catalyst | Chemical structure |
|---|---|---|
| 7 | 1,5,7-triazabicyclo[4.4.0]dec-5-ene (TBD) | 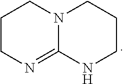 |

### Example 1.1: Synthesis of Tetrabutylphosphonium (Bisphenol A) 2, TBP-BPA₂ (CAT28)

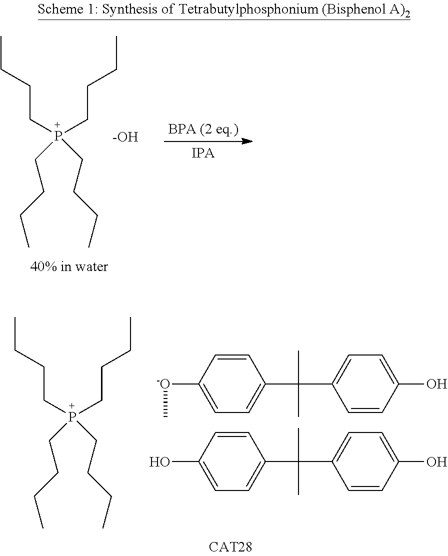

Tetrabutylphosphonium hydroxide (40% in water) (3.8 mL, 5.44 mmol) was dissolved in isopropanol (30 mL) in an RBF at room ambience. Bisphenol A (2.50 g, 10.9 mmol) was added in, and white solid started to crystallise immediately. The reaction was left to stir overnight. The mixture was then filtered, and the residue was left in the 70° C. oven to dry overnight, affording CAT28 as a white crystal (2.85 g, 73.3%). The final product was placed in the glovebox for usage.

¹H NMR (400 MHz, Methanol-d₄) δ 6.98 (d, J=8.7 Hz, 8H), 6.62 (d, J=8.7 Hz, 8H), 2.28-2.12 (m, 8H), 1.62-1.48 (m, 28H), 1.01 (t, J=7.1 Hz, 12H).

¹³C NMR (100 MHz, Methanol-d₄) δ 142.5, 128.6, 116.3, 116.2, 42.4, 31.8, 25.0 (d, J=15.8 Hz), 24.4 (d, J=4.5 Hz), 19.10 (d, J=48.0 Hz), 13.7.

³¹P NMR (162 MHz, Methanol-d₄) δ 33.5.

IR: vₘₐₓ=2965, 2930, 2871, 1612, 1592, 1510, 1467, 1444, 1378, 1353, 1273, 1230, 1173, 1098, 1080, 843 cm⁻¹.

HRMS-TOF: [M]⁺ calcd for C₁₆H₃₆P⁺ 259.25546; found 259.2546.

### Example 1.2: Synthesis of Tetrabutylphosphonium Acetate, TBP-OAc (CAT30)

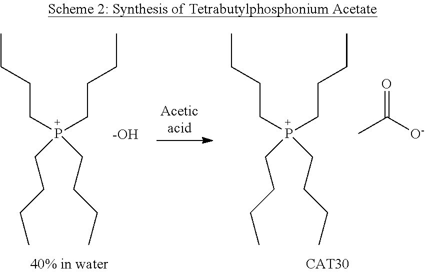

Tetrabutylphosphonium hydroxide (40% in water) (3.5 ml, 5.00 mmol) was placed in an RBF. Acetic acid (0.32 ml, 0.56 mmol) was added in and stirred overnight. Residual water was removed by reduced pressure at 80-100° C. The resulting material was dried under reduced pressure for 72 hours, affording CAT30 as a white wax material (1.4 g, 88.0%). As the crystal was highly hygroscopic, it was always kept under argon. The final product was placed in the glovebox for usage.

¹H NMR (400 MHz, Methanol-d₄) δ 2.36-2.07 (m, 8H), 1.89 (s, 3H), 1.66-1.43 (m, 16H), 1.01 (t, J=7.1 Hz, 12H).

¹³C NMR (100 MHz, Methanol-d₄) δ 180.2, 25.0 (d, J=15.3 Hz), 24.4 (d, J=4.7 Hz), 24.2, 19.1 (d, J=48.0 Hz), 13.7.

³¹P NMR (162 MHz, Methanol-d₄) δ 33.5.

IR: vₘₐₓ=2957, 2931, 2872, 1582, 1465, 1419, 1367, 1316, 1235, 1100, 920, 891 cm⁻¹.

HRMS-TOF: [M]⁺ calcd for C₁₆H₃₆P⁺ 259.25546; found 259.2551.

*Note: The mass to charge ratio of the anion is below the limit of the HRMS-TOF.

### Example 1.3: Synthesis of Tetrabutylphosphonium Butylated Hydroxytoluene, TBP-BHT (CAT32)

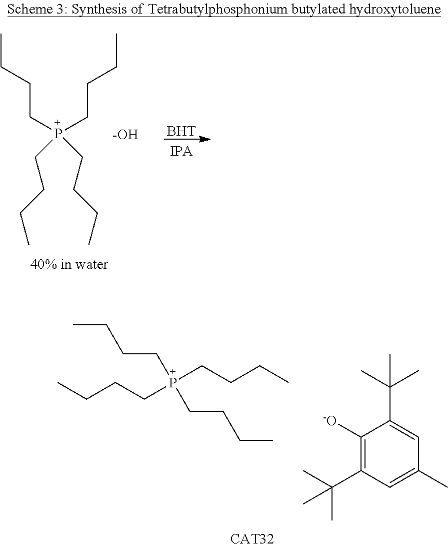

Tetrabutylphosphonium hydroxide (40% in water) (5 ml, 7.15 mmol) in isopropanol (6 mL) was purged for 3 times before adding butylated hydroxytoluene (BHT) (1.57 g, 7.15 mmol). The reaction mixture was stirred for 4 hours. The solvent was removed by vacuum stripping with ethanol (10 mL×3) at 50° C. Care was taken as CAT32 can degrade easily at high temperature. The resulting material was dried under reduced pressure, affording CAT32 as a pale-yellow solid (3.38 g, 98.9%). The final product was placed in the glovebox for usage.

¹H NMR (400 MHz, Methanol-d₄) δ 6.92 (d, J=0.8 Hz, 2H), 2.25-2.15 (m, 11H), 1.62-1.46 (m, 16H), 1.40 (s, 18H), 1.01 (t, J=7.1 Hz, 12H).

¹³C NMR (101 MHz, Methanol-d₄) δ 153.4, 139.2, 129.1, 126.1, 35.4, 31.0, 25.0 (d, J=15.3 Hz), 24.4 (d, J=4.5 Hz), 21.4, 19.0 (d, J=48.0 Hz), 13.7.

³¹P NMR (162 MHz, Methanol-d₄) δ 33.5.

IR: vₘₐₓ=2953, 2933, 2873, 2786, 1596, 1462, 1421, 1369, 1309, 1250, 1214, 1193, 1096, 925, 866 cm⁻¹.

HRMS-TOF: [M]⁺ calcd for C₁₆H₃₆P⁺ 259.25546; found 259.2545.
[M]⁻ calcd for C₁₅H₂₃O⁻ 219.17489; found 219.1747.

### Example 1.4: Synthesis of Tetrabutylphosphonium Tosylate, TBP-Tos (CAT35)

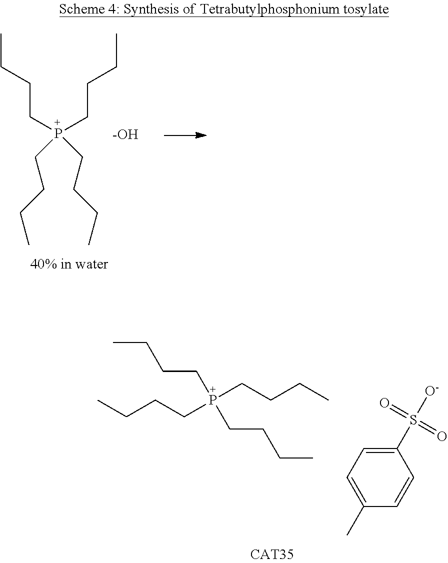

Tetrabutylphosphonium hydroxide (40% in water) (3 ml, 4.34 mmol) was dissolved in isopropanol (5 mL). p-Toluenesulfonic acid (0.83 g, 4.34 mmol) was added in and left to stir overnight. The solvent was removed by vacuum stripping with ethanol (10 mL×3) at 100° C. The resulting material was dried under reduced pressure, affording CAT35 as a white solid (0.89 g, 47.6%). The final product was placed in the glovebox for usage.

¹H NMR (400 MHz, Methanol-d₄) δ 7.71 (d, J=8.2 Hz, 2H), 7.29-7.16 (m, 2H), 2.37 (s, 3H), 2.26-2.14 (m, 8H), 1.62-1.44 (m, 16H), 1.00 (t, J=7.1 Hz, 12H).

¹³C NMR (100 MHz, Methanol-d₄) δ 143.8, 141.6, 129.8, 127.0, 25.0 (d, J=15.7 Hz), 24.4 (d, J=4.8 Hz), 21.3, 19.1 (d, J=48.1 Hz), 13.7.

³¹P NMR (162 MHz, Methanol-d₄) δ 33.4.

### Example 1.5: Synthesis of Tetrabutylphosphonium Benzoate TBP-Benzoate (CAT37)

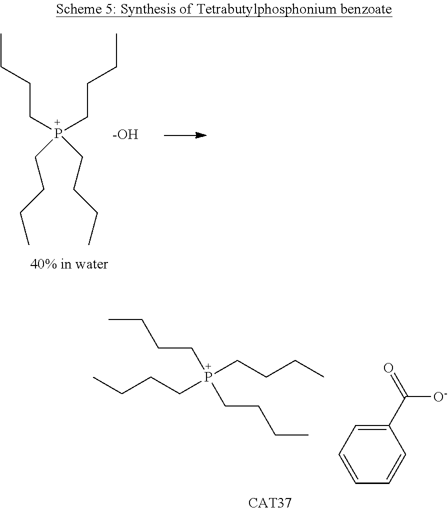

Tetrabutylphosphonium hydroxide (40% in water) (6 ml, 8.68 mmol) was placed in an RBF. Benzoic acid (1.07 g, 8.76 mmol) was added and left to stir overnight. The solvent was removed by vacuum stripping with ethanol (10 mL×3) at 100° C. The resulting material was dried under reduced pressure, affording CAT37 as a white solid (0.89 g, 47.6%). As crystal is highly hygroscopic, it is always kept under argon. The final product was placed in the glovebox for usage.

¹H NMR (400 MHz, Methanol-d₄) δ 7.96 (d, J=9.7 Hz, 2H), 7.46-7.28 (m, 3H), 2.28-2.09 (m, 8H), 1.64-1.41 (m, 16H), 1.01 (t, J=7.1 Hz, 12H).

¹³C NMR (100 MHz, Methanol-d₄) δ 175.4, 139.4, 131.2, 130.3, 128.7, 25.0 (d, J=15.3 Hz), 24.4 (d, J=4.4 Hz), 19.1 (d, J=47.9 Hz), 13.7.

³¹P NMR (162 MHz, Methanol-d₄) δ 33.5.

### Example 2: Hygroscopicity of the TBP Catalysts

The TBP catalysts synthesized in accordance to Examples 1.1 to 1.5 have different hygroscopic levels based on visual observation when the catalysts were exposed to room conditions (see Table 3). It is important to minimize the amount of water content in the catalysts as it will affect the rate of glycolysis. Hence, the catalysts were stored in the glovebox for usage.

TABLE 3
Hygroscopicity of the TBP catalysts

| No. | Catalyst | Hygroscopicity |
|---|---|---|
| 1 | CAT28 | Low |
| 2 | CAT30 | Very hygroscopic |
| 3 | CAT32 | Hygroscopic |
| 4 | CAT35 | Low |
| 5 | CAT37 | Highly hygroscopic |

### Example 3: Catalytic Activity of the TBP Catalysts

Glycolysis of polyethylene terephthalate (PET) in ethylene glycol was carried out to screen the catalysts (5 mol %). Three different PET sources were used, including commercially available PET pellets, food packaging film and contact lens film. The food packaging film (typically used for packaging yellow egg noodles) is a 3-layer PE/PET multi-layer film with a bottom linear low-density polyethylene (LLDPE), an intermediate polyurethane adhesive layer and a top PET printed layer. The contact lens film (used for packaging contact lenses) is a 5-layer metallised-PET/PE multi-layer film with a bottom linear low-density polyethylene (LLDPE), a polyurethane adhesive layer, a metallised PET intermediate layer, a polyurethane adhesive layer and a top PET printed layer.

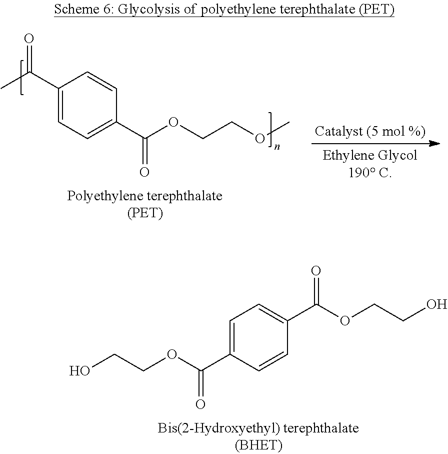

### Example 3.1: Glycolysis Using PET Pellets

Oven-dried PET pellets material (1 g, 5.2 mmol), catalyst (5 mol %, 0.26 mmol) and anhydrous ethylene glycol (4.7 mL, 5.2 g, 83.9 mmol) were added into the Schlenk tube in the glovebox. The Schlenk tube was placed into oil bath (190° C.) and stir (500 rpm) until the PET pellets fully dissolved. The time taken for full dissolution was recorded down.

For the analysis of the product distribution in the crude glycolysis reaction, NMR method with mesitylene as the internal standard was used. A known amount of mesitylene (33.3 μL) in MeOD (0.5 mL) was added to a known amount of crude (20 μL) after the glycolysis reaction and the actual percentage yield of BHET was calculated based on the integration of the NMR peaks. Most of the glycolysis reactions were carried out in duplicate to ensure the results were reproducible.

### Results and Discussion

Table 4 summarises the catalyst screening results based on the glycolysis of the PET pellets using the catalysts of the present invention, as well as the benchmark catalyst, TBD. Most catalysts afforded above 90% yield of BHET in the crude, though with some outliers and abnormalities of >100%. The abnormalities of >100% yield could be due to ethylene glycol evaporating during reaction, resulting in some errors in the calculation.

TABLE 4
Catalysts screening results based on the glycolysis of PET pellets¹

| | | | | | % |
|---|---|---|---|---|---|
| | | | | Ratio of | Yield |
| | | | Reaction | BHET to | of |
| | Catalyst | | time | dimer in the | BHET |
| Catalyst type | Code | Name | (Hrs) | product² | (IS)² |
| Benchmark | CAT4 | 1,5,7-Triazabicyclo[4.4.0]dec-5-ene | 4.00 | 96:4 | 94.7 |
| | | (TBD) | | | |
| Phosphonium | CAT26³ | Tetrabutylphosphonium hydroxide | 5.00 | 96:4 | 89.0 |
| | | | 4.00 | 96.4 | 89.0 |
| | CAT28 | Tetrabutylphosphonium BPA₂ | 3.25 | 97:3 | 97.7 |
| | CAT30 | Tetrabutylphosphonium acetate | 4.25 | 96:4 | 92.6 |
| | | | 3.50 | 96:4 | 94.5 |
| | CAT32 | Tetrabutylphosphonium butylated | 4.25 | 96:4 | >100 |
| | | hydroxytoluene | 3.25 | 96:4 | 81.2 |
| | CAT35 | Tetrabutylphosphonium tosylate | 24 | 96:4 | 91.5 |
| | CAT37 | Tetrabutylphosphonium benzoate | 3.50 | 94:6 | 76.0 |
| | | | 4.45 | 94:6 | 80.0 |
| | | | 4.25 | 95:5 | 70.0 |
| | | | 4.00 | 96:4 | 79.0 |

¹1.00 g of PET pellets, 4.7 mL of EG, 5 mol % catalyst, 190° C.
²Based on ¹H NMR analysis.
³Commercially available starting material was placed in EG and water content is removed, using ethanol, under vacuum and 50° C. water bath.

Glycolysis using benchmark catalyst TBD took 4-4.5 hours and converted PET to BHET and dimer in a ratio of 96:4. Compared to TBD, the phosphonium catalysts showed similar ratios of BHET and dimer in the products (ratio of 96:4). Using the reaction time as an indication of activity, some catalysts showed better or similar activities (i.e., 3-3.5 hours) while some showed lower activities, requiring more than 24 hours.

All the phosphonium catalysts exhibited very good activities. CAT28 and CAT32 required only 3.25 hour for complete glycolysis, while CAT30 showed a consistent 3.5-4.25 hours for its replicates. The percentage yield of BHET obtained based on the NMR analysis for CAT28, CAT30 and CAT32 were also comparable with TBD.

CAT35, with the tosylate counterpart, however, fared the worst, requiring more than 24 hours for the glycolysis reaction while there was difficulty in producing consistent results for CAT37. In conclusion, all the catalysts except CAT35 showed comparable or even better performance than the benchmark catalyst TBD.

CAT28 and CAT30 catalysts were identified to be effective catalysts for glycolysis using PET pellets. CAT32 is effective but degrades easily during synthesis.

### Example 3.2: Glycolysis Using Food Packaging Film

Oven-dried food packaging film (1 g), catalyst (5 mol %, 0.26 mmol) and anhydrous ethylene glycol (4.7 mL, 5.2 g, 83.9 mmol) was added into the Schlenk tube in the glovebox. The Schlenk tube was placed into oil bath (190° C.) and stir (500 rpm) until the food packaging film fully dissolved. The time taken for full dissolution was recorded down.

### Results and Discussion

Table 5 summarises the catalyst screening results based on the glycolysis of the food packaging film using the catalysts of the present invention, as well as the benchmark catalyst, TBD. Compared to TBD, most of the catalysts showed similar ratios of BHET and dimer in the products (ratio of 96:4). Using the reaction time as an indication of activity, as compared to TBD at 45 minutes, some catalysts showed better or similar activities i.e., 40-45 minutes, while some showed lower activities, requiring 50-55 minutes.

TABLE 5
Catalysts screening results based on the glycolysis of food packaging film¹

| | | | | Ratio of | | |
|---|---|---|---|---|---|---|
| | | | Reaction | BHET to | BHET | |
| Catalyst | Catalyst | | time | dimer in the | % | Reaction |
| type | Code | Name | (min) | product² | (IS)² | yield %³ |
| Benchmark | CAT4 | 1,5,7-Triazabicyclo | 45 | 95:5 | 72 | 77 |
| | | [4.4.0]dec-5-ene (TBD) | 45 | 94:6 | 69 | 73 |
| Phosphonium | CAT28 | Tetrabutyl | 45 | 95:5 | 71 | 75 |
| | | phosphonium BPA₂ | 50 | 94:6 | 75 | 80 |
| | CAT30 | Tetrabutyl | 40 | 94:6 | 74 | 79 |
| | | phosphonium acetate | 45 | 96:4 | 77 | 82 |
| | CAT32 | Tetrabutyl | 55 | 96:4 | 78 | 83 |
| | | phosphonium butylated | 45 | 96:4 | 77 | 82 |
| | | hydroxytoluene | | | | |
| | CAT37 | Tetrabutylphosphonium | 40 | 96:4 | 68 | 72 |
| | | benzoate | 50 | 96:4 | 70 | 74 |

¹1.00 g of food packaging film, 4.7 mL of EG, 5 mol % catalyst, 190° C. for 1 hour. The time for full dissolution is noted.
²Yield based on 1 g of film, inclusive of insolubles, using ¹H NMR analysis with internal standard
³Yield excluding insolubles (insolubles ~0.060 g)

The BHET %, in 1 g of film after reaction, range from 69-78%. This scenario was expected, due to the presence of other components, such as adhesive, additives and dye pigments in the film. The reaction yield ranged between 72-82%, excluding the insolubles of 60 mg.

Overall, results of CAT28 and CAT30 for food packaging film look promising and are comparable with the benchmark catalyst TBD.

### Example 3.3: Glycolysis Using Contact Lens Film

Glycolysis reaction of contact lens film was carried out using the benchmark catalyst and the TBP catalysts the same way as the food packaging film.

### Results and Discussion

Table 6 summarises the glycolysis results using contact lens film based on the catalysts of the present invention, as well as the benchmark catalyst, TBD. Compared to TBD, most of the catalysts showed similar ratios of BHET and dimer in the products (ratio of 96:4). Using the reaction time as an indication of activity, as compared to TBD at 30 minutes, some catalysts showed similar activities i.e., 30-35 minutes, while some showed lower activities, requiring 45-50 minutes.

TABLE 6
Catalysts screening results based on the glycolysis of contact lens film¹

| | | | | Ratio of | | |
|---|---|---|---|---|---|---|
| | | | Reaction | BHET to | BHET | |
| Catalyst | Catalyst | | time | dimer in the | % | Reaction |
| type | Code | Name | (mins) | product² | (IS)² | yield %³ |
| Benchmark | CAT4 | 1,5,7-Triazabicyclo | 30 | 94:6 | 64 | 71 |
| | | [4.4.0]dec-5-ene (TBD) | 30 | 93:7 | 60 | 67 |
| Phosphonium | CAT28 | Tetrabutyl | 40 | 93:7 | 55 | 61 |
| | | phosphonium BPA₂ | 40 | 91:9 | 58 | 64 |
| | CAT30 | Tetrabutyl | 35 | 90:10 | 59 | 66 |
| | | phosphonium acetate | 35 | 92:8 | 61 | 68 |
| | CAT32 | Tetrabutyl | 40 | 93:1 | 64 | 71 |
| | | phosphonium butylated | 45 | 92:8 | 61 | 68 |
| | | hydroxytoluene | | | | |
| | CAT37 | Tetrabutylphosphonium | 50 | 96:4 | 72 | 80 |
| | | Benzoate | 45 | 96:4 | 68 | 75 |

¹1.00 g of Contact Lens film, 4.7 mL of EG, 5 mol % catalyst, 190° C. for 1 hour. The time for full dissolution is noted.
²Yield based on 1 g of film, inclusive of insolubles, using ¹H NMR analysis with internal standard
³Yield excluding insolubles (insolubles ~0.100 g).

The BHET % yield, in 1 g of film after reaction, ranged from 55-72%, lower than the food packaging film. This scenario was expected, due to the presence of higher amount of insolubles (~0.1 g) and other components, such as adhesive, additives and dye pigments in the film. The reaction yield ranged between 60-70%, excluding the insolubles.

Overall, results of CAT30 for contact lens film look promising and are comparable with the benchmark TBD.

### Example 4: Thermal Stability of the TBP Catalysts

The thermal stability of the catalyst used for glycolysis is crucial as the reaction is carried out at a high temperature of 190° C. The thermal stability of the catalysts can be investigated using ³¹P NMR analysis, due to the phosphorus moiety, and ¹H NMR analysis for both the cation and anion moiety of the catalysts. Thermal stability studies for CAT28, CAT30 and CAT37 were carried out.

Catalysts CAT28 and CAT30: 0.15 g of catalyst in 1.0 mL of EG under argon was heated at 195° C. for 4 hours, and then at 210° C. for 1 hour and finally at 230° C. for 1 hour in a pressure tube. Aliquots of the sample were taken out in the glovebox at each temperature point and then submitted for ³¹P NMR and ¹H NMR analysis in DMSO-de.

Catalyst CAT37: 0.15 g of catalyst in 1.0 mL of EG under argon was heated at 230° C. for 3 hours in a pressure tube. Aliquots of the sample were taken out in the glovebox and then submitted for ³¹P NMR and ¹H NMR analysis in DMSO-de. There was no colour change for CAT37, even at high temperature of 230° C.

Benchmark TBD: 0.15 g of TBD in 1.0 mL of EG under argon was heated at 195° C. for 4 hours, and then at 210° C. for 1 hour and finally at 230° C. for 1 hour in a pressure tube. Aliquots of the sample were taken out in the glovebox at each temperature point and then submitted for ¹H NMR analysis in MeOH-d₄.

### Results and Discussion

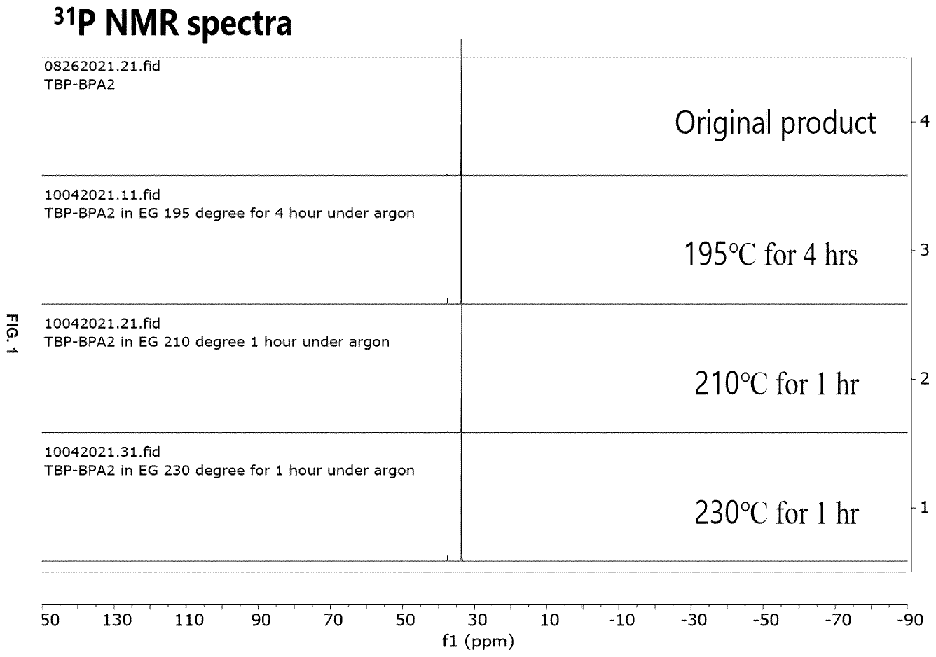

Catalyst CAT28: There was a gradual change in colour from colourless to brown when the sample was heated at higher temperature and prolonged hours. There is no evident decomposition of the cation portion even at 230° C. for 1 hour as seen from the ³¹P NMR spectra (FIG. 1).

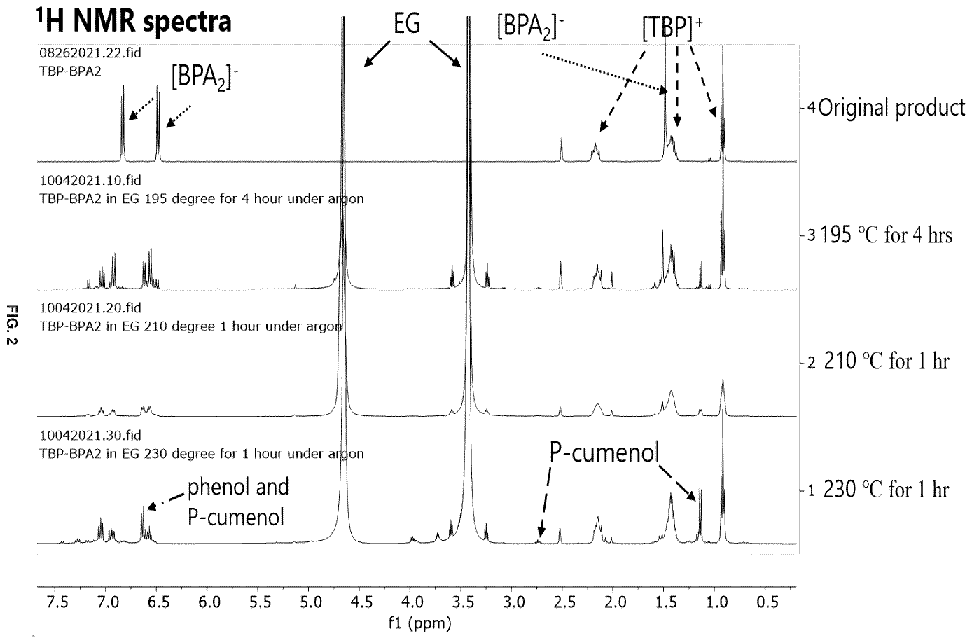

Similarly, as shown in ¹H NMR analysis (FIG. 2), there is no evident decomposition of the cation part. However, at 195° C. for 4 hours, part of the anion moiety BPA₂ can be seen decomposing to phenol and p-cumenol (Scheme 7). There was total conversion at 230° C. for 1 hour. But interestingly, the anion of phenol and p-cumenol were stable up to 290° C. and this should not impair the catalyst's performance.

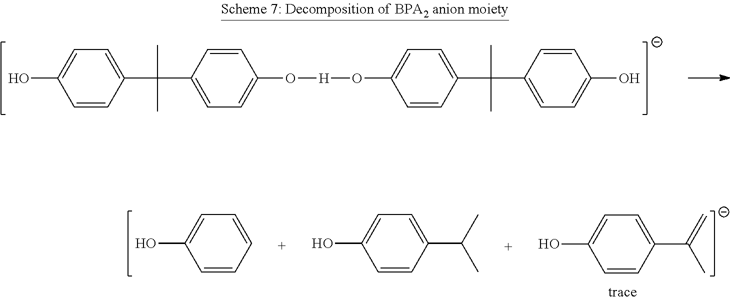

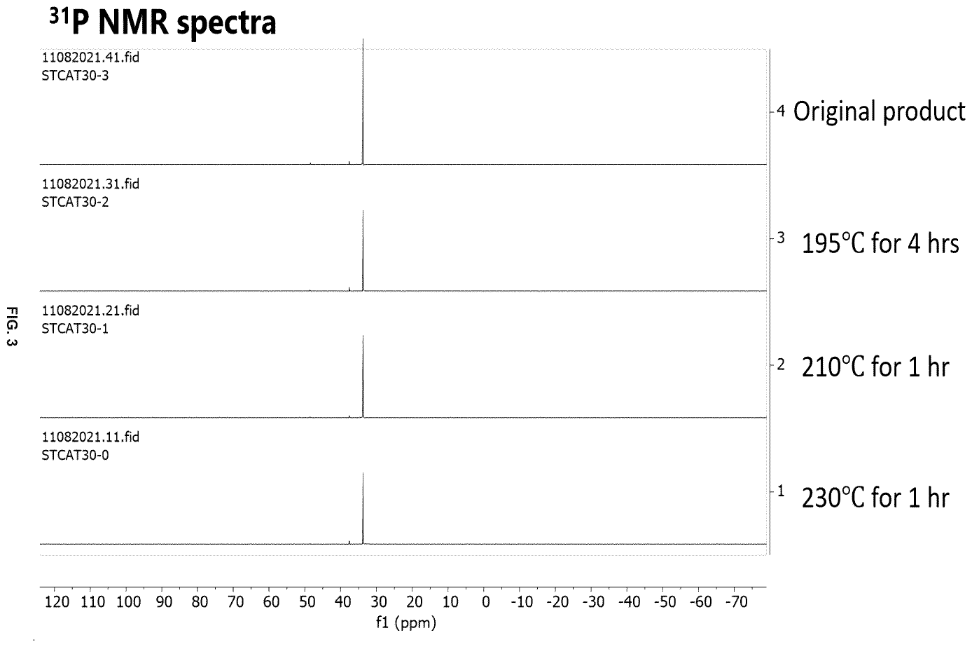

Catalyst CAT30: There was no colour change for CAT30, even at high temperature of 230° C. There is no evident decomposition of the cation portion even at 230° C. for 1 hour as seen from the ³¹P NMR spectra in FIG. 3.

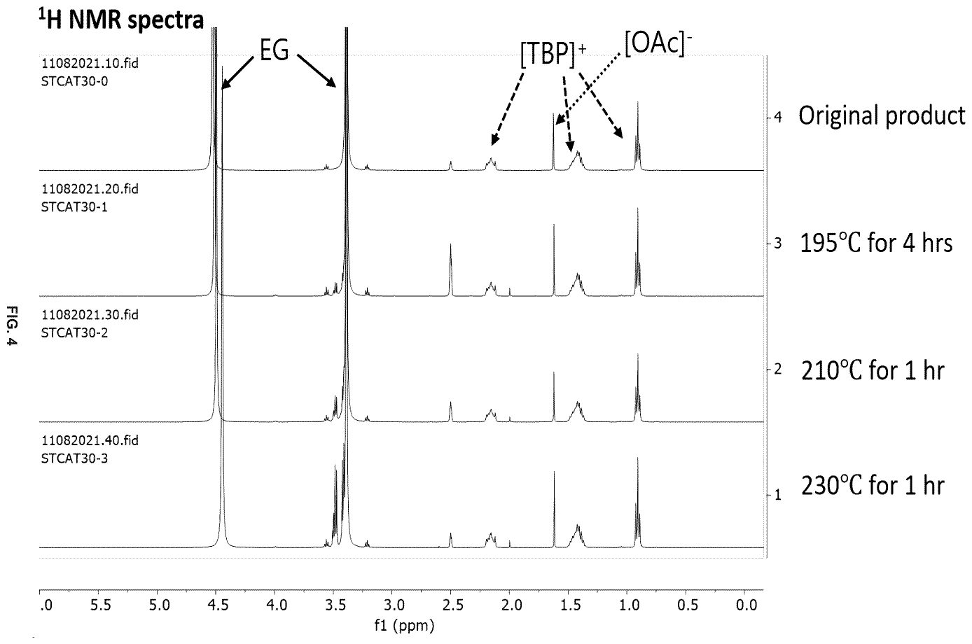

¹H NMR analysis in FIG. 4 shows that there is no evident decomposition of the cation part. At higher temperature of 210° C., some degradation can be seen. This could be due to the small presence of acetic acid present in the catalyst, due to excess acetic acid used in the catalyst synthesis, causing dehydration of ethylene glycol, forming diethylene glycol, shown in Scheme 8.

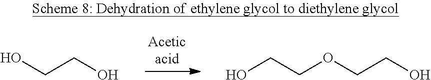

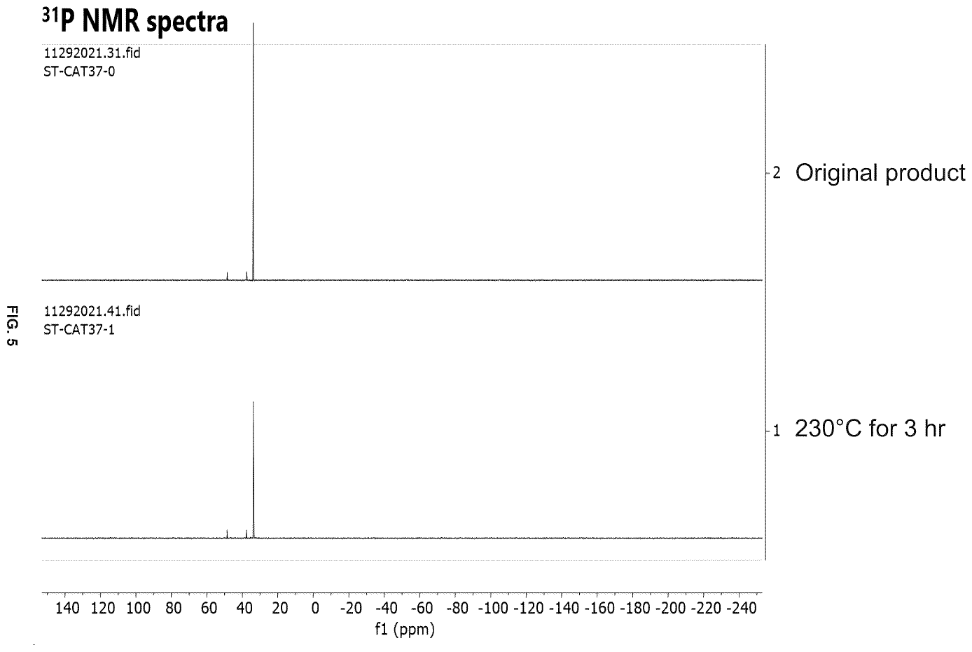

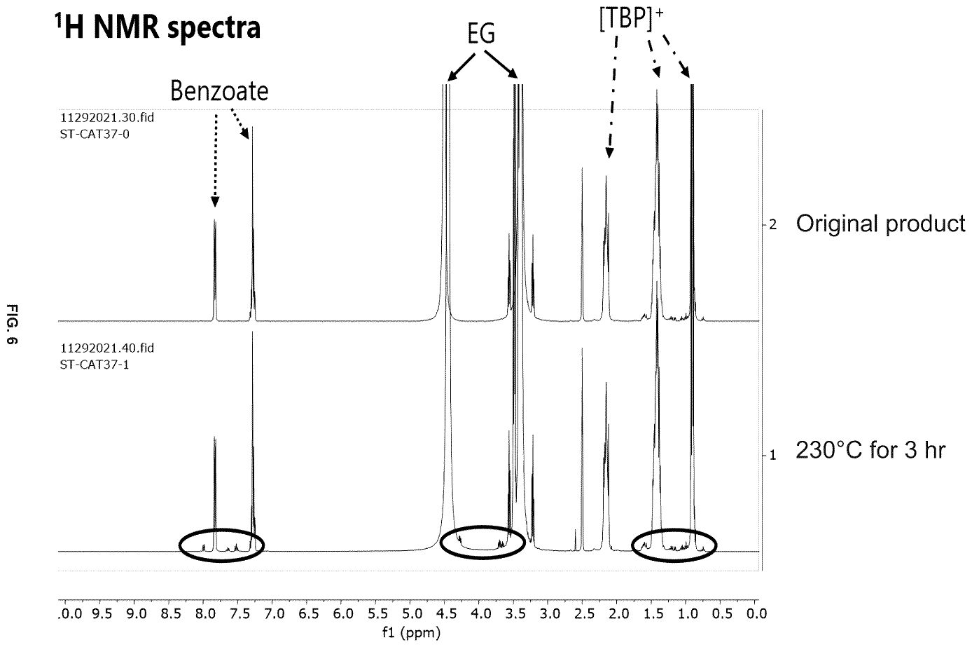

Catalyst CAT37: As shown in both 1H and ³¹P NMR analysis in FIG. 5 and FIG. 6, there is no evident decomposition of the cation or the anion part of CAT37 after heating at 230° C. for 3 hours. CAT37 is highly thermal stable.

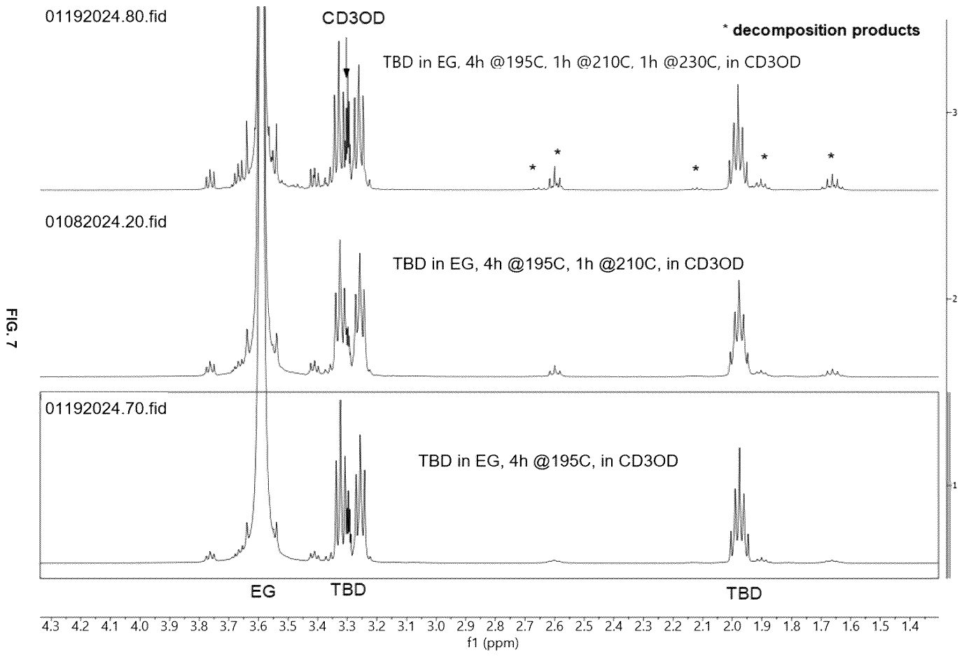

Benchmark TBD: There was a change in colour from colourless to light brown when the sample was heated at 230° C. As shown in the ¹H NMR analysis in FIG. 7, there is evident decomposition of TBD at 230° C. New aliphatic peaks in the range from 1.6 to 2.7 ppm integrate to approximately 30% relative to the CH₂ from TBD at 1.98 ppm.

Results of the thermal stability studies are summarised in Table 7.

TABLE 7
Results of catalysts screening based on thermal stability

| Catalyst | | Testing | End | |
|---|---|---|---|---|
| Code | Name | Temp/° C. | Colour | Remarks |
| Benchmark | 1,5,7-Triazabicyclo | 195 | Light | Stable with significant |
| | [4.4.0]dec-5-ene (TBD) | 210 | Brown | decomposition. |
| | | 310 | | |
| CAT28 | Tetrabutylphosphonium | 195 | Brown | Cation part is stable while |
| | BPA2 | 210 | | anion part gradually |
| | | 230 | | decomposed to other |
| | | | | stable anions |
| CAT30 | Tetrabutylphosphonium | 195 | Colourless | Cation and anion parts are |
| | acetate | 210 | | stable with minimal |
| | | 230 | | deposition at 230° C. |
| CAT37 | Tetrabutylphosphonium | 230 | Colourless | Cation and anion parts are |
| | benzoate | | | stable with minimal |
| | | | | deposition at 230° C. |

1. A process for the solvolysis of a polyester, the process comprising:
(a) mixing the polyester with a solvolysis reagent and a catalyst, wherein the catalyst comprises:
a cation of formula Ia: 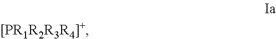
wherein each of R₁, R₂, R₃ and R₄ is independently selected from a linear or branched C₁ to C₂₀ alkyl group; or
wherein R₁ and R₂ and/or R₃ and R₄ together form an alkylene chain —(CH₂)ₚ— where p is from 3 to 6, such that R₁ and R₂ and/or R₃ and R₄ form, together with the phosphorous atom that they are attached to, a cyclic ring; and
an anion; and
(b) heating the mixture to a predetermined temperature.

2. The process according to claim 1, wherein each of R₁, R₂, R₃ and R₄ is independently a linear or branched C₂ to C₁₀ alkyl group, such as a linear or branched C₃ to C₅ alkyl group.

3. The process according to claim 1, wherein the cation is a tetrabutylphosphonium ion.

4. The process according to claim 1, wherein the cation is

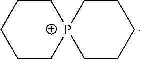 [SMILES: C1CC[P+]2(CC1)CCCCC2]

5. The process according to claim 1, wherein the anion is selected from the group consisting of a hydroxide, a halide, an alkoxide, a phenoxide, a carboxylate, a carbonate, a bicarbonate and a sulfonate.

6. The process according to claim 5, wherein:
(ai) when the anion is a halide, the halide is selected from chloride and bromide;
(aii) when the anion is an alkoxide, the alkoxide has a formula IIa: 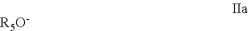
wherein R₅ is a linear or branched C₁ to C₆ alkyl group;
(aiii) when the anion is a phenoxide, the phenoxide has a formula IIb: 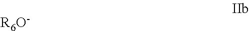
wherein R₆ is an aryl group which is unsubstituted or substituted by one or more linear or branched C₁ to C₄ alkyl groups,
each the linear or branched C₁ to C₄ alkyl group is unsubstituted or substituted by a second aryl group which is unsubstituted or substituted by a hydroxyl group, optionally wherein the phenoxide forms a hydrogen bond with a second protonated phenoxide to form a dimer;
(aiv) when the anion is a carboxylate, the carboxylate has a formula IIc: 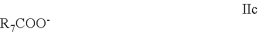
wherein R₇ is selected from a linear or branched C₁ to C₆ alkyl group and an aryl group which is unsubstituted or substituted by one or more substituents selected from a linear or branched C₁ to C₄ alkyl group and a carboxylate group, optionally wherein the anion is p-terephthalate;
(av) when the anion is a sulfonate, the sulfonate has a formula IId: 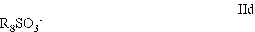
wherein R₅ is selected from a linear or branched C₁ to C₆ alkyl group and an aryl group which is unsubstituted or substituted by one or more linear or branched C₁ to C₄ alkyl groups.

7. The process according to claim 1, wherein the anion is selected from the group consisting of:

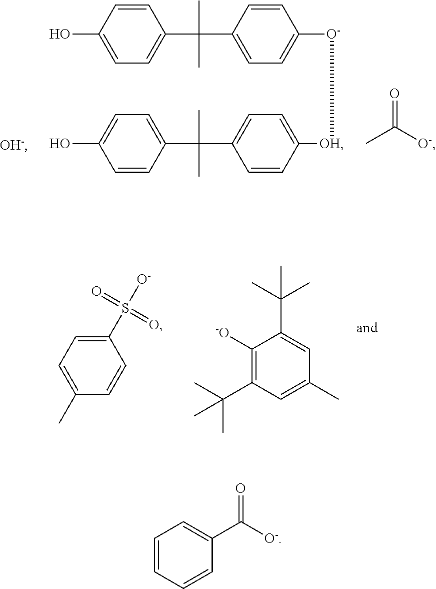

8. The process according to claim 1, wherein the anion is selected from the group consisting of:

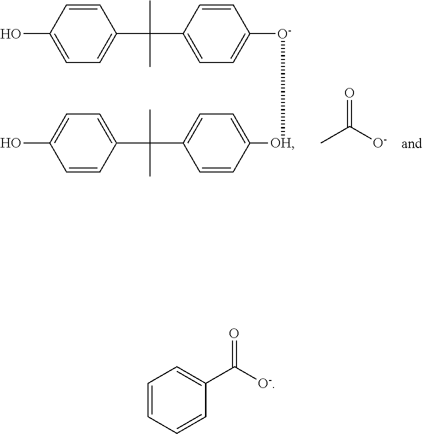

9. The process according to claim 1 wherein the predetermined temperature is from 180 to 240° C.

10. The process according to claim 1, wherein the solvolysis reagent is selected from the group consisting of water, an alcohol, an amine and ammonia, optionally wherein the alcohol is selected from a group consisting of methanol, ethanol, sorbitol 1,3-propanediol, 1,4-butanediol, 1,6-hexanediol and ethylene glycol.

11. The process according to claim 1, wherein the solvolysis reagent is ethylene glycol.

12. The process according to claim 1, where in the polyester is a aliphatic polyester or aromatic polyester,
optionally wherein the aromatic polyester is selected from the group consisting of polyethylene terephthalate e (PET), polybutylene terephthalate (PBT), polyhexamethylene terephthalate (PHT), polypropylene terephthalate (PPT).

13. The process according to claim 1, wherein the polyester is polyethylene terephthalate (PET).

14. A catalyst for the solvolysis of a polyester, the catalyst comprises:
a cation of formula Ia: 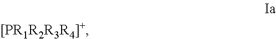
wherein R₁, R₂, Ra and R₄ are independently a linear or branched C₁ to C₂₀ alkyl group; or
wherein R₁ and R₂ and/or R₃ and R₄ together form an alkylene chain —(CH₂)ₚ— where p is from 3 to 6, such that R₁ and R₂ and/or R₃ and R₄ form, together with the phosphorous atom that they are attached to, a cyclic ring; and
an anion,
wherein the anion is selected from the group consisting of an alkoxide, a phenoxide, a carbonate, a bicarbonate and a sulfonate.

15. The catalyst according to claim 14, wherein:
(a) when the anion is an alkoxide, the alkoxide has a formula IIa: 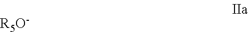
wherein R₅ is a linear or branched C₁ to C₆ alkyl group;
(b) when the anion is a phenoxide, the phenoxide has a formula IIb: 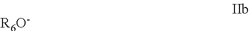
wherein Rₑ is an aryl group which is unsubstituted or substituted by one or more linear or branched C₁ to C₄ alkyl groups,
each the linear or branched C₁ to C₄ alkyl group is unsubstituted or substituted by a second aryl group which is unsubstituted or substituted by a hydroxyl group, optionally wherein the phenoxide forms a hydrogen bond with a second protonated phenoxide to form a dimer;
(c) when the anion is a sulfonate, the sulfonate has a formula IId: 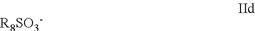
wherein R₅ is selected from a linear or branched C₁ to C₆ alkyl group and an aryl group which is unsubstituted or substituted by one or more linear or branched C₁ to C₄ alkyl groups.

16. The catalyst according to claim 14, wherein the anion is selected from the group consisting of:

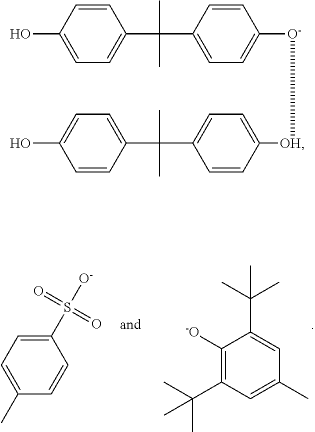

17. The catalyst according to claim 14, wherein the anion is

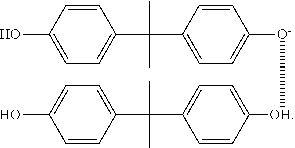

18. The catalyst according to claim 14, wherein each of R₁, R₂, R₃ and R₄ is independently a linear or branched C₂ to C₁₀, such as a linear or branched C₃ to C₈ alkyl group.

19. The catalyst according to claim 14, wherein the cation is a tetrabutylphosphonium ion.

20. The catalyst according to claim 14, wherein the cation is 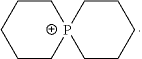 [SMILES: C1CC[P+]2(CC1)CCCCC2]
